(19) United States
(12) Patent Application Publication
(10) Pub. No.: US 2003/0190323 A1
(43) Pub. Date: Oct. 9, 2003

(54) Preparations and methods for the treatment of T cell mediated diseases

(75) Inventors: Irun R. Cohen, Rehovot, IL (US); Dana Elias, Gedera, IL (US); Meir Shinitzky, Kfar Shmaryahu, IL (US)

Correspondence Address: WINSTON & STRAWN, PATENT DEPARTMENT, 1400 L STREET, N.W., WASHINGTON, DC, 20005-3502, US

(73) Assignee: YEDA RESEARCH AND DEVELOPMENT CO. LTD.

(21) Appl. No.: 10/307,326

(22) Filed: Dec. 2, 2002

Related U.S. Application Data
(63) Continuation-in-part of application No. 08/981,861, filed on Apr. 19, 1998, now Pat. No. 6,488,933.
Application No. 08/981,861 is a 371 of international application No. PCT/US96/11373, filed on Jul. 2, 1996.

(30) Foreign Application Priority Data
Jul. 5, 1995 (IL) 114,458

(51) Int. Cl. A61K 39/00; A61K 35/78

(52) U.S. Cl. 424/185.1; 424/757

(57) ABSTRACT

The present invention provides pharmaceutical compositions and methods for reducing the incidence or severity of insulin dependent diabetes mellitus, insulitis, β-cell destruction, or latent autoimmune diabetes in adults by administering to a patient a pharmaceutical composition comprising an antigen and a carrier, wherein the antigen is recognized by inflammatory T cells associated with the pathogenesis of the disease and the carrier is a metabolizable lipid emulsion. The composition induces a TH1→TH2 shift in the cytokines produced by said T cells and it is administered in an amount, which is therapeutically effective to reduce the symptoms of the disease.

### CROSS REFERENCE

This application is a continuation-in-part of U.S. patent application Ser. No. 08/981,861, filed Apr. 19, 1998, which is a 371 national stage application of PCT/US96/11373, filed Jul. 2, 1996.

### FIELD OF THE INVENTION

The present invention relates to compositions and methods of reducing the severity of insulin-dependent diabetes mellitus or insulitis. The invention further relates to compositions and methods of reducing or inhibiting β-cell destruction. The invention further relates to compositions and methods of reducing the severity of latent diabetes. More generally, the invention relates to vaccine therapy for T-cell mediated diseases, and in particular to therapeutic preparations comprising peptides recognized by T cells involved in the pathogenesis of T cell mediated diseases, such as autoimmune diseases, and a metabolizable lipid emulsion as a biologically active carrier.

### BACKGROUND OF THE INVENTION

Autoimmune disorders, e.g., insulin-dependent diabetes mellitus (IDDM or type I diabetes), multiple sclerosis, rheumatoid arthritis and thyroiditis, are characterized by reactivity of the immune system to an endogenous antigen, with consequent injury to tissues. These immune responses to self-antigens are maintained by the persistent activation of self-reactive T lymphocytes.

T cells of the CD4 “helper” type have been divided into two groups by the characteristic cytokines they secrete when activated (Mosmann and Coffman, 1989). THI cells secrete IL-2, which induces T cell proliferation, and cytokines such as IFN-γ, which mediate tissue inflammation. TH2 cells, in contrast, secrete IL-4 and IL-10. IL-4 helps T cells secrete antibodies of certain IgG isotypes and suppresses the production of THI inflammatory cytokines (Banchereau et al., 1994). IL-10 indirectly inhibits THI activation by affecting antigen-presentation and inflammatory cytokine production by macrophages (Moore et al., 1993). It is the THI cells which contribute to the pathogenesis of organ-specific autoimmune diseases. TH1-type responses also appear to be involved in other T cell mediated diseases or conditions, such as contact dermatitis (Romagnani, 1994).

Peptides suitable for immunologically specific therapy of an autoimmune disease are peptides that are recognized by T cells involved in the pathogenesis of the autoimmune disease. Each autoimmune disease will have its ideal peptide for use in therapy. A disease like multiple sclerosis involving T cells reactive to self-antigens such as myelin basic protein (MBP) (Allegreta et al., 1990) will require a peptide of myelin basic protein for its therapy, as for example those described by Ota et al., 1990.

The present inventors have shown that autoimmune diseases such as type I diabetes mellitus may be treated by administering a suitable peptide in an oil vehicle. Non-obese diabetic (NOD) mice spontaneously develop type I diabetes caused by autoimmune T cells that attack the insulin-producing β cells of the islets. The autoimmune attack is associated with T-cell reactivity to a variety of self-antigens including a peptide of the 60 kDa heat shock protein (hsp 60) and peptides of glutamic acid decarboxylase (GAD). Thus, for example, spontaneous diabetes developing in the NOD/Lt strain of mice could be treated with a peptide designated p277 corresponding to positions 437-460 of the human hsp 60 sequence (PCT Patent Publication No. W090/10449; D. Elias and I. R. Cohen, Peptide therapy for diabetes in NOD mice, The Lancet 343:704-06, 1994); with variants of the p277 peptide in which one or both cysteine residues at positions 6 and/or 11 have been replaced by valine and/or the Thr residue at position 16 is replaced by Lys (see PCT Publication W096/19236) and with peptides designated p12 and p32 corresponding to positions 166-185 and 466-485, respectively, of the human hsp60 sequence. See PCT Publication WO 97/01959 of the same applicant of the present application, the entire contents of which are hereby incorporated by reference.

Another type of diabetes, known as Latent Autoimmune Diabetes in Adults (LADA) may be treated by administering p277 or analogs thereof. The term LADA has been introduced to define non-insulin requiring adult diabetes patients with immune markers of type 1 diabetes. Like classic type 1 diabetes patients in children and adults, LADA patients have similar genetic predisposition to diabetes, the same diabetes-associated antibodies, similar insulitis and show a progression to severe insulin deficiency. Initially, a proportion of non-insulin dependent diabetes mellitus (NIDDM) patients present symptoms resembling type 2 diabetic patients such as insulin resistance and absence of HLA antigens, however, these patients will progress to insulin therapy and have HLA genes and immune changes normally associated with type I diabetes, consistent with having an immunologically mediated disease process, which damages insulin secreting cells. Recent studies suggest that about 10-20% of NIDDM patients have this autoimmune process, therefore the prevalence of this subtype of diabetes may be comparable or even higher than the prevalence of type 1 diabetes. About 80% of recently diagnosed NIDDM patients with autoimmune markers progress to insulin requirement within 3-6 years.

For patients in whom the primary defect is loss of beta cell function it follows that treatment should logically aim to restore beta cell mass or function. Prevention of progression of NIDDM towards insulin therapy has raised considerable interest. At present, aside from immunotherapy, such treatment might include the use of sulphonylureas or insulin. While insulin therapy could be valuable in maintaining beta cell function it would be illogical to use insulin in a study aiming to alter the risk of progression to insulin treatment and dependence. Moreover, it has been recently revealed that administration of insulin to people at high-risk for type 1 diabetes had no effect on the loss of beta-cell function or on the progression to insulin dependency. Sulphonylureas are already used extensively in patients with NIDDM and while their efficacy has not been formally tested it is evident that their use has not arrested the progression to insulin dependency.

Peptide therapy for treatment of IDDM using p12, p32, p277 or variants thereof, was found by the present inventors to be effective when the peptide was administered to mice subcutaneously (sc) in an oil vehicle such as an emulsion of mineral oil known as incomplete Freund's adjuvant (IFA). However, IFA as well as complete Freund's adjuvant (CFA; a preparation of mineral oil containing various amounts of killed organisms of Mycobacterium) are not allowed for human use because the mineral oil is not metabolizable and cannot be degraded in the body. Therefore, it would be desirable to discover an effective vehicle for peptide therapy of IDDM and LADA that would be metabolizable.

Several fat emulsions have been in use for many years for intravenous nutrition of human patients. Two of the available commercial fat emulsions, known as INTRALIPID®, a registered trade mark of Kabi Pharmacia, Sweden, for a fat emulsion for intravenous nutrition described in U.S. Pat. No. 3,169,094 and LIPOFUNDIN® (a registered trade mark of B. Braun Melsungen, Germany) contain soybean oil as fat (100 or 200 g in 1,000 ml total preparation: 10% or 20%, respectively). Egg-yolk phospholipids (6-12 g/l distilled water) are used as emulsifiers in INTRALIPID® and in LIPOFUNDIN®. Isotonicity results from the addition of glycerol (22-25 g/l ) in INTRALIPID® and in LIPOFUNDIN®. These fat emulsions are quite stable and have been used for total parenteral nutrition of patients suffering from gastrointestinal or neurological disorders, which prevent them from receiving nutrition orally, and thus they receive the calories needed to sustain life. Usual daily doses are of up to 1 liter daily.

U.S. Pat. No. 4,073,943 issued on Feb. 14, 1978 to Wretlind et al. and Re. 32,393 issued on May 29, 1990 as reissue patent of U.S. Pat. No. 4,168,308 issued on Sep. 18, 1979 to Wretlind et al., describe a carrier system for use in enhancing parenteral, particularly intravenous, administration of a pharmacologically active, oil-soluble agent, comprising a stable, oil-in-water emulsion containing a pharmacologically inert lipoid as a hydrophobic phase dispersed in a hydrophilic phase, said lipid being dispersed in the emulsion as finely divided particles having a mean particle size less than 1 micron to achieve rapid onset of an acceptable therapeutic effect, said carrier system being used with an effective dose of said pharmacologically active, oil-soluble agent predominantly dissolved in said lipoid at a fraction ratio thereto in the hydrophobic phase, said therapeutic effect being attributable to said effective dose of the active agent. This carrier system is said to be suitable for administration of a water-insoluble or water-soluble, oil-soluble pharmacologically active agent that is predominantly dissolved in the lipoid phase. Examples of such pharmacologically active agents are depressants, anaesthetics, analgesics, stimulants, spasmolytics, muscle relaxants, vasodepressants and diagnostic, e.g. X-ray contrast, agents. The carrier system is said to enhance the diagnostic or therapeutic effect of the agent with a rapid onset accompanied by a reduced incidence of injury to body tissues.

INTRALIPID® has been proposed as a non-irritating vehicle for several adjuvants for use in vaccines such as, for example, 6-0-(2-tetradecylhexadecanoyl)- and 6-0-(3-hydroxy-2docosylhexacosanoyl) N-acetylmuramyl-L-alanyl-D-isoglutamine (Tsujimoto et al., 1986 and 1989), avridine (Woodard and Jasman, 1985), N,N-dioctadecyl-N′,N′-bis(2-hydroxyethyl) propanediamine (CP-20,961) (German Patent Application No. DE 2945788; Anderson and Reynolds, 1979; Niblack et al., 1979). Kristiansen and Sparrman, 1983, have disclosed that the immunogenicity of hemagglutinin and neuraminidase in mice is markedly increased after adsorption onto lipid particles constituting INTRALIPID®.

None of the above publications describe the use of INTRALIPID® as a vehicle for peptides in the treatment of autoimmune diseases, nor has there been any disclosure that INTRALIPID® could serve as a tolerogenic vehicle for a therapeutic antigen in order to mediate a shift of the immune response from a TH1-type response to a TH2-type response.

### SUMMARY OF THE INVENTION

The invention relates to compositions and methods of reducing the incidence or severity of insulin dependent diabetes mellitus (IDDM) or insulitis. The invention further relates to compositions and methods of reducing or inhibiting β-cell destruction. The invention further relates to compositions and methods of reducing the severity of latent autoimmune diabetes in adults (LADA).

The present invention relates to compositions used for the treatment of IDDM or insulitis or β-cell destruction or LADA, comprising a peptide or other antigen and a biologically active lipid carrier, wherein the peptide or other antigen is one recognized by inflammatory T-cells associated with the pathogenesis of said disease or condition, and the biologically active lipid carrier is a fat emulsion comprising about 5-25% triglycerides of plant and/or animal origin, about 0.1-3% phospholipids of plant and/or animal origin, about 1.5-4.5% osmo-regulator, and optionally about 0-0.5% antioxidant, and sterile water to complete to 100%.

The triglycerides and phospholipids of plant or animal origin may derive from any suitable vegetable oil, such as soybean oil, cottonseed oil, coconut oil, or olive oil, or from egg-yolk or bovine serum. Preferably, the triglycerides are derived from soybean oil and the phospholipids are derived from soybean oil or from egg-yolk. Typically the triglycerides and phospholipids are present in a weight ratio of between about 3:1 and 50:1, and preferably between about 5:1 and 25:1, respectively. More preferably, however, the triglyceride/phospholipid ratio is between about 10:1 and 20:1.

Any suitable osmo-regulator may be added to the fat emulsion, preferably glycerol, xylitol or sorbitol. The fat emulsion may optionally include an anti-oxidant, for example 0.05% alpha-tocopherol.

In one embodiment of the invention, the fat emulsion as defined above is processed by centrifugation, e.g. at 10,000 g or higher, thus forming a small triglyceride-rich (about 90% triglycerides) layer on the top of a phospholipid enriched aqueous dispersion containing about 1:1 triglycerides:phospholipids, and this latter aqueous dispersion is used as the lipid vehicle in the preparations of the invention.

Typically, phosphatidylcholine may constitute 40% to 70% of the phospholipid content in the lipid emulsion depending on the source of the phosphatidylcholine; if derived from plant source, phosphatidylcholine may constitute 40-50% of the phospholipids, and if derived from animal source it may constitute 60-70% of the phospholipids. As alternative embodiments, it is possible to use individual isolated phospholipids such as phosphatidylcholine or phosphatidylethanolamine or any other suitable phospholipid species.

The present invention also provides methods of reducing the severity of IDDM or insulitis or β-cell destruction or LADA comprising administering to a patient said disease, a pharmaceutical composition of an antigen or a peptide and a carrier, wherein the antigen is recognized by inflammatory T cells associated with the pathogenesis of said disease and the carrier is a metabolizable lipid emulsion, said composition induces a TH1→TH2 shift in the cytokines produced by said T cells. Typically the composition is administered in an amount sufficient to reduce the severity of symptoms associated with said disease or to halt or slow the progression of the disease. Preferably, the composition is administered in an amount sufficient to reduce the severity of symptoms associated with IDDM or to halt or slow the progression of IDDM.

Advantageously, the composition is administered in an amount sufficient to cause a decrease in IL-2 or IFN-γ TH1-cell cytokine response and an increase in IL-4 or IL-10 TH2-cell cytokine response.

It is preferable that the pharmaceutical composition be administered to the patient prior to complete β-cell destruction and more preferable that the composition be administered prior to the loss of 50-75% β-cell function.

A non-limiting example of a preferred embodiment is wherein the pharmaceutical composition used comprises peptide p277 (residues 437-460 of SEQ ID NO: 1) or peptide p277 (Val⁶-Val¹¹) (SEQ ID NO:4) or other peptide selected from Table 1 below.

Furthermore, derivatives of these peptides can also be used as long as the derivatives are recognized by the inflammatory T cells associated with the pathogenesis of IDDM and/or β-cell destruction. One skilled in the art may use the examples set forth herein to develop further antigens and derivatives of disclosed antigens to be used with the methods disclosed herein. It is preferable that the derivatives of peptide p277 (residues 437-460 of SEQ ID NO: 1) or peptide p277 (Val⁶-Val¹¹) (SEQ ID NO:4) used be at least 70% homologous, more preferably at least 85% homologous, and most preferably 95% homologous to the amino acid sequences of residues 437-460 of SEQ ID NO: 1 or SEQ ID NO: 4.

It is preferable that the emulsion be a fat or lipid emulsion comprising about 5-25% triglycerides, about 0.1-3% phospholipids, about 1.5-4.5% osmo-regulator, and sterile water to complete to 100%, and more preferably the emulsion comprises about 8-20% triglycerides, about 0.24-2.4% phospholipids, about 2-4% osmo-regulator, and water, and still more preferably, the emulsion comprises about 10-20% triglycerides, about 0.6-1.2% phospholipids, about 2.2 to 2.5% osmo-regulator and water. Optionally, about 0-0.05% antioxidant can also be added to the emulsion. The triglycerides and phospholipids used are of plant and/or animal origin.

Examples of exemplary emulsions contain 10% soybean oil, 0.6-1.2% egg-yolk phospholipids, 2.2-2.5% glycerol and sterile water.

It has now been found that metabolizable lipid emulsions, such as those described above as well as the preferred embodiments thereof, namely INTRALIPID® and LIPOFUNDIN®, can be successfully used as vehicles for peptide therapy of autoimmune diseases and of other TH1 T cell mediated diseases or conditions. It has been further found that this activity is associated with a TH1 to TH2 cytokine shift.

In one preferred embodiment of the invention, the preparation is for the treatment of insulin-dependent diabetes mellitus (IDDM) and comprises a peptide derived from the human heat shock protein 60 (hsp60) that is recognized by inflammatory T-cells associated with the pathogenesis of IDDM, wherein said peptide is selected from the group of peptides appearing in the following Table 1.

In another preferred embodiment of the invention, the preparation is for the treatment of latent autoimmune diabetes in adults (LADA) and comprises a peptide derived from the human heat shock protein 60 (hsp60) that is recognized by inflammatory T-cells associated with the pathogenesis of diabetes, wherein said peptide is selected from the group of peptides appearing in the following Table 1.

The fat emulsions of the present invention are preferably used as freshly prepared or after storage in a container, which is not open to the atmospheric air.

TABLE 1

| | | Amino acid sequence |
|---|---|---|
| Peptides | Sequence ID No: | (one letter code) |
| p13 | 1 (31-50) | KFGADARALMLQGVDLLADA |
| p10 | 1 (136-155) | NPVEIRRGVMLAVDAVIAEL |
| p11 | 1 (151-170) | VIAELKKQSKPVTTPEEIAQ |
| p12 | 1 (166-185) | EEIAQVATISANGDKEIGNI |
| p14 | 1 (195-214) | RKGVITVKDGKTLNDELEII |
| P18 | 1 (255-274) | QSIVPALEIANAHRKPLVIIA |
| p20 | 1 (286-305) | LVLNIRLKVGLQVVAVKAPGF |
| p24 | 1 (346-365) | GEVIVTKIDDAMLLKGKGDKA |
| p29 | 1 (421-440) | VTDALNATRAAVEEGIVLGG |
| p30 | 1 (436-455) | IVLGGGCALLRCIPALDSLT |
| p32 | 1 (466-485) | EIIKRTLKIPAMTIAKNAGV |
| p135 | 1 (511-530) | VNMVEKGIIDPTKVVRTALL |
| p39 | 1 (343-366) | GKVGEVIVTKDDAM |
| p277 | 1 (437-460) | VLGGGCALLRCIPALDSLTPANED |
| p277 (Val⁶) | * 2 | VLGGGVALLRCIPALDSLTPANED |
| p277 (Val¹¹) | ** 3 | VLGGGCALLRVIPALDSLTPANED |
| p277 (Val⁶-Val¹¹) | *** 4 | VLGGGVALLRVIPALDSLTPANED |

*437-460 of SEQ ID NO: 1 with C-442 changed to V
**437-460 of SEQ ID NO: 1 with C-447 changed to V
***437-460 of SEQ ID NO: 1 with C-442 and C-447 changed to V

### BRIEF DESCRIPTION OF THE DRAWINGS

FIG. 1 shows anti-p277 antibody production in NOD mice treated with the peptide p277(Val⁶-Val¹¹) in: (i) INTRALIPID® or (ii) phosphate-buffered saline (PBS), as described in Example 2.

FIG. 2 shows TH2-dependent antibody isotypes induced in NOD mice by treatment with the peptide p277(Val⁶-Val¹¹) in INTRALIPID®, as described in Example 3 below.

FIGS. 3A-B show that p277(Val⁶-Val¹¹)/INTRALIPID® therapy induces in NOD mice a specific switch in the profile of cytokines produced by the T-cells reactive to the p277(Val⁶-Val¹¹) peptide, as described in Example 4. FIG. 3A shows that there is a reduction of TH1 (IL-2, IFN-γ) and elevation of TH2 (IL-4, IL-10) cytokines after treatment of the mice with the p277(Val⁶-Val¹¹) peptide in INTRALIPID® and incubation of the spleen cells with p277(Val⁶-Val¹¹); FIG. 3B shows that there is no change in the cytokines after treatment of the mice with the p277(Val⁶-Val¹¹) peptide in INTRALIPID® and incubation of the spleen cells with Con A.

FIG. 4 shows that spontaneous T-cell proliferative responses to p277(Val⁶-Val¹¹) is reduced after treatment with the p277(Val⁶-Val¹¹) peptide in INTRALIPID®, as described in Example 5.

FIG. 5 shows that treatment of rats with myelin basic protein, peptide p71-90 in INTRALIPID® reduces the severity of experimental autoimmune encephalomyelitis (EAE), as described in Example 6.

FIG. 6 shows that treatment of rats with myelin basic protein peptide p71-90 in IFA reduces the severity of experimental autoimmune encephalomyelitis (EAE), as described in Example 6.

### DETAILED DESCRIPTION OF THE INVENTION

The present invention relates to compositions and methods of reducing the incidence or severity of insulin dependent diabetes mellitus (IDDM) or insulitis and further relates to compositions and methods of reducing or inhibiting β-cell destruction. The present invention further relates to compositions and methods of reducing the severity of latent autoimmune diabetes in adults (LADA).

The present invention relates to compositions used for the treatment of IDDM or insulitis or β-cell destruction or LADA, comprising a peptide or other antigen and a biologically active lipid carrier, wherein the peptide or other antigen is recognized by inflammatory T-cells associated with the pathogenesis of said disease or condition, and the biologically active lipid carrier is a fat emulsion comprising about 5-25% triglycerides of plant and/or animal origin, about 0.1-3% phospholipids of plant and/or animal origin, about 1.5-4.5% osmo-regulator, and optionally about 0-0.5% antioxidant, and sterile water to complete to 100%.

The present invention also provides methods of reducing the severity of IDDM or insulitis or β-cell destruction or LADA comprising administering to a patient having said disease a pharmaceutical composition of an antigen or a peptide and a carrier, wherein the antigen is recognized by inflammatory T cells associated with the pathogenesis of said disease and the carrier is a metabolizable lipid emulsion, said composition induces a TH1→TH2 shift in the cytokines produced by said T cells. Typically the composition is administered in an amount sufficient to reduce the severity of symptoms associated with said disease or to halt or slow the progression of the disease.

A preferred method comprises administering to a patient having IDDM or at risk of developing IDDM, a pharmaceutical composition of an antigen and a carrier, wherein the antigen is recognized by inflammatory T cells associated with the pathogenesis of diabetes and the carrier is a metabolizable lipid emulsion, said composition induces a TH1→TH2 shift in the cytokines produced by said T cells. Typically the composition is administered in an amount sufficient to reduce the severity of symptoms associated with IDDM or to halt or slow the progression of IDDM.

Advantageously the composition is administered in an amount sufficient to cause a decrease in IL-2 or IFN-γ TH1-cell cytokine response and an increase in IL-4 or IL-10 TH2-cell cytokine response.

Generally the pharmaceutical composition is administered to the patient prior to complete β-cell destruction and more preferable that the composition be administered prior to the loss of 50-75% β-cell function.

A preferred embodiment is one wherein the pharmaceutical composition used comprises peptide p277 (residues 437-460 of SEQ ID NO: 1) or peptide p277 (Val⁶-Val¹¹) (SEQ ID NO: 4) or other peptide selected from Table 1 above. Furthermore, derivatives of these peptides can also be used as long as the derivatives are recognized by the inflammatory T cells associated with the pathogenesis of IDDM and/or β-cell destruction and/or LADA. One skilled in the art may use the examples set forth herein to develop further peptides and derivatives of disclosed peptides to be used with the methods disclosed herein. It is preferable that the derivatives of peptide p277 (residues 437-460 of SEQ ID NO: 1) or peptide p277 (Val⁶-Val¹¹) (SEQ ID NO: 4) used be at least 70% homologous, more preferably at least 85% homologous, and most preferably 95% homologous to the amino acid sequences of residues 437-460 of SEQ ID NO: 1 or SEQ ID NO: 4.

Because of the relative shortness of the p277 peptide, 24 residues, chemical or chain elongation synthesis is presently felt to be the method of choice. Analogs of p277 having one or more substitutions can be readily synthesized in this manner and then tested for biological activity in a straightforward manner to determine the specific biological effect of such substitution(s).

The term “homology” is used in its usual and well known sense of indicating correspondence between members in an amino acid (AA) sequence. For purposes of this application, the term homologous refers to at least about 70% correspondence, the term substantially homologous refers to a correspondence of at least about 85%, and the term highly homologous refers to a correspondence of at least about 90% or preferably about 95% or higher.

The term “analog” includes any peptide having an amino acid residue sequence generally identical to a sequence specifically shown herein, e.g., p277 (residues 437-460 of SEQ ID NO: 1), wherein one or more residues has been replaced (with at least about 80% and preferably at least about 85%, and more preferably 90% of the residues being the same, with at least 95% or higher being the most preferred) or additional amino acid residues have been added or modified and wherein the analog displays the ability to biologically mimic the parent molecule as described herein in some particular function. Preferably, most if not all of such substitutions are replacements of a residue with a functionally similar residue, i.e. conservative substitutions. Examples of such conservative substitutions include: the substitution of one non-polar (hydrophobic) residue, such as isoleucine, valine, alanine, glycine, leucine or methionine for another non-polar residue; the substitution of one polar (hydrophilic) residue for another polar residue, such as arginine for lysine, glutamine for asparagine, threonine for serine; the substitution of one basic residue such as lysine, arginine or histidine for another basic residue; and the substitution of one acidic residue, i.e. aspartic acid or glutamic acid, for the other. The phrase “conservative substitution” is also intended to include the use of a chemically derivatized residue in place of a non-derivatized residue provided that the resultant polypeptide displays the requisite biological activity, e.g. binding activity. For purposes of this application, two peptides are considered to be substantially the same when they only differ from each other by conservative substitutions.

It is preferable that the emulsion used with the antigen comprise about 5-25% triglycerides, about 0.1-3% phospholipids, about 1.5-4.5% osmo-regulator, and the balance being water, and more preferably the emulsion comprises about 8-20% triglycerides, about 0.24-2.4% phospholipids, about 2-4% osmo-regulator, and water, and still more preferably the emulsion comprises about 10-20% triglycerides, about 0.6-1.2% phospholipids, about 2.2 to 2.5% osmo-regulator and water. Optionally, about 0-0.05% antioxidant can also be added to the emulsion. The triglycerides and phospholipids used are of plant and/or animal origin.

Examples of exemplary emulsions contain 10% soybean oil, 0.6-1.2% egg-yolk phospholipids, 2.2-2.5% glycerol and sterile water. Typically, the pharmaceutical composition is administered to the patient as soon after being clinically diagnosed as having IDDM as possible. Additionally, however, the pharmaceutical composition may be administered to a patient that has not yet developed IDDM but is prone to developing it. Furthermore, the pharmaceutical composition may be administered to a patient suffering from insulitis or suspected as suffering from any type of β-cell destruction.

The pharmaceutical compositions of the invention can further be administered to a patient as a preventive measure to groups prone to developing IDDM prior to actually being diagnosed with clinical symptoms of IDDM. With this option of the invention, the patient can be identified as having a high risk of developing IDDM by genetic test, by study of the medical history of his family or by performing assays to determine the functional β-cell mass. The pharmaceutical composition can then be administered to those patients identified as being prone to IDDM.

The pharmaceutical compositions may be administered to a patient suffering from LADA. Alternatively, the pharmaceutical composition may be administered to a patient that has not yet developed LADA but is prone to developing it.

In one non-limiting example, explained in more detail below, it was found that P277(Val⁶-Val¹¹)-peptide treatment, in an appropriate carrier, was able to down-regulate the spontaneous T-cell proliferative responses to epitopes of both hsp60 and GAD and abolished the production of autoantibodies to hsp60, to GAD and to insulin. It was found that by administering a pharmaceutical composition comprising at least P277(Val⁶-Val¹¹)-peptide to a patient that the severity of symptoms associated with IDDM and the chances of the patient developing full IDDM were minimized. It was found that the arrest of the disease process was associated, not with T-cell tolerance or anergy, but with a shift in the cytokines produced by the autoimmune T cells reactive to p277(Val⁶-Val¹¹) from a TH1-like profile (IL-2, IFNγ) to a TH2-like profile (IL-4, IL-10). The modulation was immunologically specific; the spontaneous T-cell response of the treated mice to a bacterial hsp60 peptide remained in the TH1 mode. Thus, the diabetogenic process characterized by autoimmunity to several self antigens can be cured using one of the antigens, e.g., peptide p277(Val⁶-Val¹¹).

The association of p277(Val⁶-Val¹¹) therapy with a switch in reactivity to p277(Val⁶-Val¹¹) from T-cell proliferation to antibodies indicates that the therapeutic effect results from a shift in the predominant cytokines produced by the autoimmune T cells in the treated mice. TH1 cells secrete IL-2, which induces T-cell proliferation, and cytokines such as IFN-γ, which mediate tissue inflammation, thereby contributing to the pathogenesis of the disease; TH2 cells, in contrast, secrete IL-4 and IL-10. IL-4 helps B cells secrete antibodies of certain IgG isotypes and suppresses the production of TH1 inflammatory cytokines. IL-10 indirectly inhibits TH1 activation by affecting antigen-presentation and inflammatory cytokine production by macrophages. Thus, TH2 cells suppress TH1 activity (see Liblau et al., 1995). The shift from TH1 to TH2-like behavior was supported by analysis of the isotypes of the antibodies produced before and after p277(Val⁶-Val ¹¹) therapy.

The fact that the mechanism of the therapeutic effect of the peptide in a lipid vehicle treatment is shown to involve a TH1→TH2 cytokine shift, provides the possibility of using the TH1→TH2 shift as evidence that the treatment was effective and did induce a beneficial response. In other words, the TH1→TH2 shift can serve as a surrogate marker of the response to treatment. For example, the lack of the shift can indicate a need for a second treatment. See PCT Publication WO 97/01959, the entire contents of which are hereby incorporated herein by reference. As per ongoing clinical trial results, ongoing therapy with multiple administrations of the compositions of the present invention may be required.

The lipid emulsions of the present invention, when used as a vaccine adjuvant with the antigenic substance to which the T cells involved in the disease or condition being treated are active, serve to mediate a shift from a TH1 T cell response prior to treatment to a TH2 T cell response after treatment. This finding establishes that such lipid emulsions are tolerogenic biologically active carriers which can be used in vaccines for the treatment of any TH1 mediated disease or condition. In such vaccines, the antigen provides the immunological specificity for a therapeutic effect while the biologically active carrier and the antigen of the present invention provide the biological outcome, i.e., the TH1→TH2 shift. Because of the shift mediated by said biologically active carrier of the present invention, diseases with a spectrum of autoreactivities can be turned off with a single antigen/carrier combination capable of inducing a T cell cytokine shift.

A preferred use in accordance with the present invention is in the treatment of organ-specific autoimmune diseases, which are mediated by TH1 cells. Such diseases include, but are not limited to, autoimmune diseases such as IDDM, rheumatoid arthritis, multiple sclerosis and thyroiditis. The peptide used in such treatment is an autoantigen peptide. Thus, for example, for IDDM the peptide is the above-mentioned p277 peptide or the valine substituted analog p277(Val⁶-Val¹¹); for multiple sclerosis such peptide is derived from myelin basic protein; for thyroiditis the peptide is thought to be derived from thyroglobulin, and for rheumatoid arthritis the autoantigen can derive from Mycobacterium organisms, e.g., Mycobacterium tuberculosis.

It is not critical that the antigen be a peptide. Thus, for example, TH1-mediated allergic responses which result in skin sensitivity and inflammation, such as contact dermatitis, can be treated by a vaccine containing the irritant antigen and a biologically active carrier in accordance with the present invention which will cause a shift in the cytokine response from a TH1-type to a TH2-type. Thus, while the patient will continue to have elevated antibody levels against the antigen, the inflammatory T cell response causing the skin irritation will be suppressed.

Accordingly, the tolerogenic biologically active carrier of the present invention may be used any time that it is desired to create tolerance for the antigen which the cells are attacking, i.e., any time that a immunotherapy is being used to restrict a T cell mediated condition, particularly a TH1 cell mediated condition. If it can be determined which antigen is activating the response in graft rejection or in graft-versus-host disease, then the administration of such an antigen with a carrier in accordance with the present invention would be expected to facilitate the shift of the undesirable inflammatory TH1response to a more desirable TH2 response, regardless of the overall complexity of the number of antigens to which T cells are active in such condition.

To determine the T-cell secretion of cytokines following activation with peptides, lymphocytes from the peripheral blood of patients are tested in an in vitro activation assay. Peripheral blood lymphocytes are isolated from whole heparinized blood on ficol-hypaque, and cultured with the test peptide(s) at concentrations of 5-50 μg/ml. The supernatants from the cultured T-cells are collected at different time points and tested for activity of various cytokines, by ELISA or bioassay(s).

Examples of fat emulsions that can be used in the preparations of the present invention preferably include, but are not limited to, the commercially available INTRALIPID® and LIPOFUNDIN® for intravenous nutrition, and the fat emulsions described in the above-mentioned U.S. Pat. Nos. 3,169,096, 4,073,943 and 4,168,308, herein incorporated by reference in their entirety. However, the finding according to the present invention that these metabolizable lipids, administered previously for intravenous nutrition, may be used effectively as vehicles for therapy of T cell mediated diseases, is completely unexpected. Similarly, the discovery that these preparations are tolerogenic biologically active carriers which mediate a TH1→TH2 shift is also totally unexpected.

The fat emulsions of the present invention are preferably used as freshly prepared or after storage in a container which is not open to the atmospheric air. Prolonged storage of INTRALFPID®, for example, while exposed to atmospheric air, causes a decrease in the pH and a corresponding decrease in the biological activity.

In one preferred embodiment, the biologically active carrier of the invention is a fat emulsion comprising 10% soybean oil, 0.6-1.2% egg-yolk phospholipids, 2.2-2.5% glycerol and sterile water to complete to 100 ml (INTRALIPID® 10%). In another embodiment, the vehicle is a fat emulsion comprising 20% soybean oil, 1.2% egg yolk phospholipids, 2.2-2.5% glycerol and sterile water to complete to 100 ml.

Other preferred embodiments of the carrier comprise: 10% soybean oil; 0.8% phospholipids (egg); 2.5% glycerol; 0.05% alpha-Tocopherol; and sterile water; 20% soybean oil; 1.2% phospholipids (egg); 2.5% glycerol; 0.05% alpha-Tocopherol; and sterile water; 10% soybean oil; 0.6%-1.2% phospholipids (egg yolk); 2.2% glycerol; and sterile water; and 20% soybean oil; 1.2% phospholipids (egg yolk); 2.2% glycerol; and sterile water.

In yet another preferred embodiment, the vehicle is a fat emulsion comprising 5% soybean oil and 5% safflower oil, 0.6-1.2% egg-yolk phospholipids, 2.5% glycerol and distilled water to complete to 100 ml (LIPOFUNDIN® 10%).

In one embodiment of the invention, the vehicle is a processed lipid emulsion obtained by centrifugation, e.g. at 10,000 g or higher, of the original fat emulsion defined herein, whereby a small triglyceride-rich (about 90% triglycerides) is formed on the top of a phospholipid-enriched aqueous dispersion containing about 1:1 triglycerides:phospholipids. The two phases are separated and the phospholipid-rich aqueous dispersion is used as the vehicle.

The preparations of the invention may comprise one or more peptides. Thus, for example, for the treatment of IDDM, the preparation may comprise one or more of the peptides p12, p32, p277, p277 (Val⁶), p277 (Val¹¹), p277 (Val⁶-Val¹¹), or any of the other peptides of Table 1. The peptide can be prepared using any of the fat emulsion disclosed herein, however, a preferred embodiment would include a peptide p277 or p277(Val⁶-Val¹¹) and a fat emulsion comprising 10% soybean oil, 0.6-1.2% egg-yolk phospholipids, 2.2-2.5% glycerol and sterile water to complete to 100 ml (INTRALIPID® 10%). Optionally, about 0.05% antioxidant can also be added to the emulsion.

The invention further relates to the use of a fat emulsion as defined herein or of a processed phospholipid enriched aqueous dispersion prepared therefrom by centrifugation for the preparation of a therapeutic preparation comprising one or more peptides or other antigens and said fat emulsion or processed aqueous dispersion as a vehicle in the therapy of autoimmune diseases or other TH1 mediated diseases or conditions.

Typically, the phosphatidylcholine may constitute 40% to 70% of the phospholipid content in the lipid emulsion depending on the source of the phosphatidylcholine; if derived from plant source, phosphatidylcholine may constitute 40-50% of the phospholipids, and if derived from animal source it may constitute 60-70% of the phospholipids. As alternative embodiments, it is possible to use individual isolated phospholipids such as phosphatidylcholine, phosphatidylethanolamine, phosphatidylserine, phosphatidy inositol or any other suitable phospholipid species.

### EXAMPLES

The invention will now be illustrated by the following non-limiting examples.

### Example 1

Peptide Therapy of Type I Diabetes Using p277(Val⁶-Val¹¹) in Oils

The efficacy of various lipid preparations as vehicles for peptide therapy of the diabetes of NOD mice was tested. In this model, autoimmune destruction of the insulin producing β-cells in the pancreas is mediated by T-lymphocytes. An inflammatory infiltrate develops around the pancreatic islets at 5-8 weeks of age and β-cell destruction leading to insulin deficiency and overt diabetes becomes manifested at 14-20 weeks of age affecting almost look of female NOD mice by 35-40 weeks of age.

NOD female mice were treated with 100 μg of peptide p277(Val⁶-Val¹¹) per mouse sc in 0.1 ml of: (i) Phosphate-buffered saline (PBS), or (ii) a 10% lipid emulsion composed of 10% soybean oil, 0.6-1.2% egg phospholipids and 2.2-2.5% glycerol (INTRALIPID®, Kabi Pharmacia AB, Sweden).

The incidence of diabetes at 6 months of age and the production of anti-p277(Val⁶-Val¹¹) antibodies was followed. Diabetes was diagnosed as persistent hyperglycemia, blood glucose levels over 11 mmol/L measured at least twice at weekly intervals with a Beckman Glucose Analyzer II. Successful peptide treatment was assayed by maintenance of a normal blood glucose concentration (less than 11 mmol/L), remission of the intra-islet inflammation of the pancreatic islets (insulitis) and induction of antibodies to the therapeutic peptide as an indicator of a TH2-type immune response. The results are shown in Table 2.

TABLE 2
Incidence of Diabetes at 6 months

| Treatment | | |
|---|---|---|
| incidence (%) | Diabetes | Death (%) |
| p277 (Val⁶-Val¹¹)/PBS | 90 | 80 |
| p277 (Val⁶-Val¹¹)/INTRALIPID ® | 45# | 20# |
| none | 100 | 90 |

# p<0.01 compared to untreated NOD mice.

As can be seen from Table 2, peptide treatment administered in INTRALIPID® was effective in reducing the incidence of diabetes and death. On the other hand, treatment administered in PBS was ineffective.

### Example 2

Anti-p277(Val⁶-Val¹¹) Antibody Production

The protection from diabetes by treatment with the p277(Val⁶-Val¹¹) peptide is dependent on TH2 immunological reactivity to the peptide. Therefore, antibody production was measured in the p277(Val⁶-Val¹¹)-immunized mice by ELISA. Maxisorp microtiter plates (Nunc) were coated with p277(Val⁶-Val¹¹) peptide, 10 μg/ml, for 18 h and non-specific binding blocked with 7% milk powder for 2 h. The mouse sera, diluted 1:50, were allowed to bind for 2 h and the specific binding was detected by adding alkaline phosphatase anti-mouse IgG (Serotec) for 2 h and p-nitrophenylphosphate substrate (Sigma) for 30 min. The color intensity was measured by an ELISA reader (Anthos) at OD=405 nm.

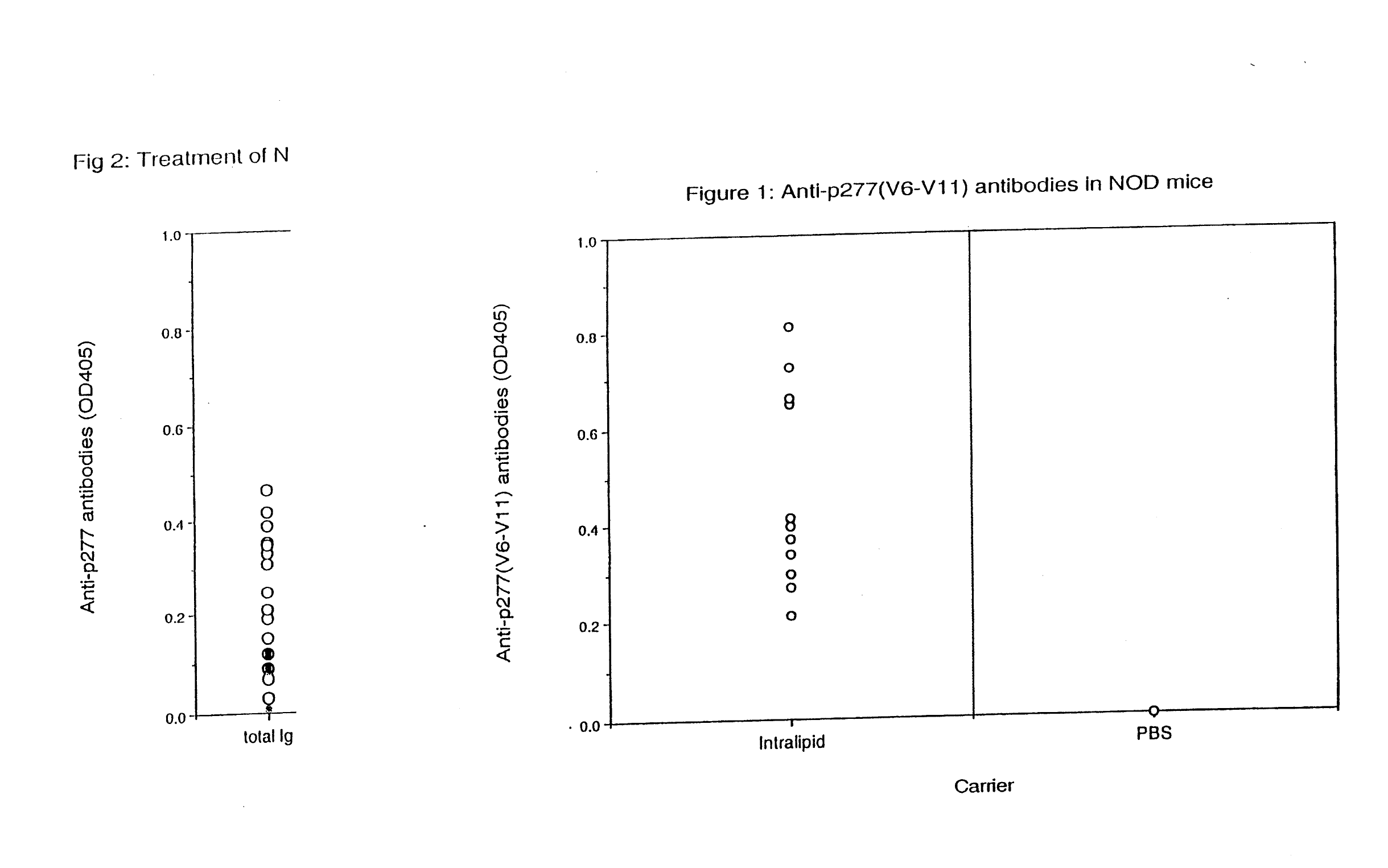

As can be seen from FIG. 1, NOD mice immunized to p277(Val⁶-Val¹¹) in INTRALIPID® developed peptide specific antibodies, while mice immunized to p277(Val⁶-Val¹¹) in PBS showed no antibody responses at all.

### Example 3

Antibody Isotypes Induced by p277(Val⁶-Val¹¹) Therapy

The association of p277(Val⁶-Val¹¹) INTRALIPID® therapy with antibodies to p277(Val⁶-Val¹¹) shown in Example 2, suggested that the therapeutic effect might result from a shift in the predominant cytokines produced by the autoimmune T cells. T cells of the CD4 “helper” type have been divided into two groups by the characteristic cytokines they secrete when activated (Mosmann and Coffman, 1999): TH1 cells secrete IL-2, which induces T-cell proliferation, and cytokines such as IFNγ, which mediate tissue inflammation; TH2 cells, in contrast, secrete IL-4, which “helps” B cells produce certain antibody isotypes, and IL-10 and other cytokines, which can “depress” tissue inflammation. The possibility of a shift from TH1 to TH2-like behavior was supported by analysis of the isotypes of the antibodies produced after p277(Val⁶-Val¹¹) therapy.

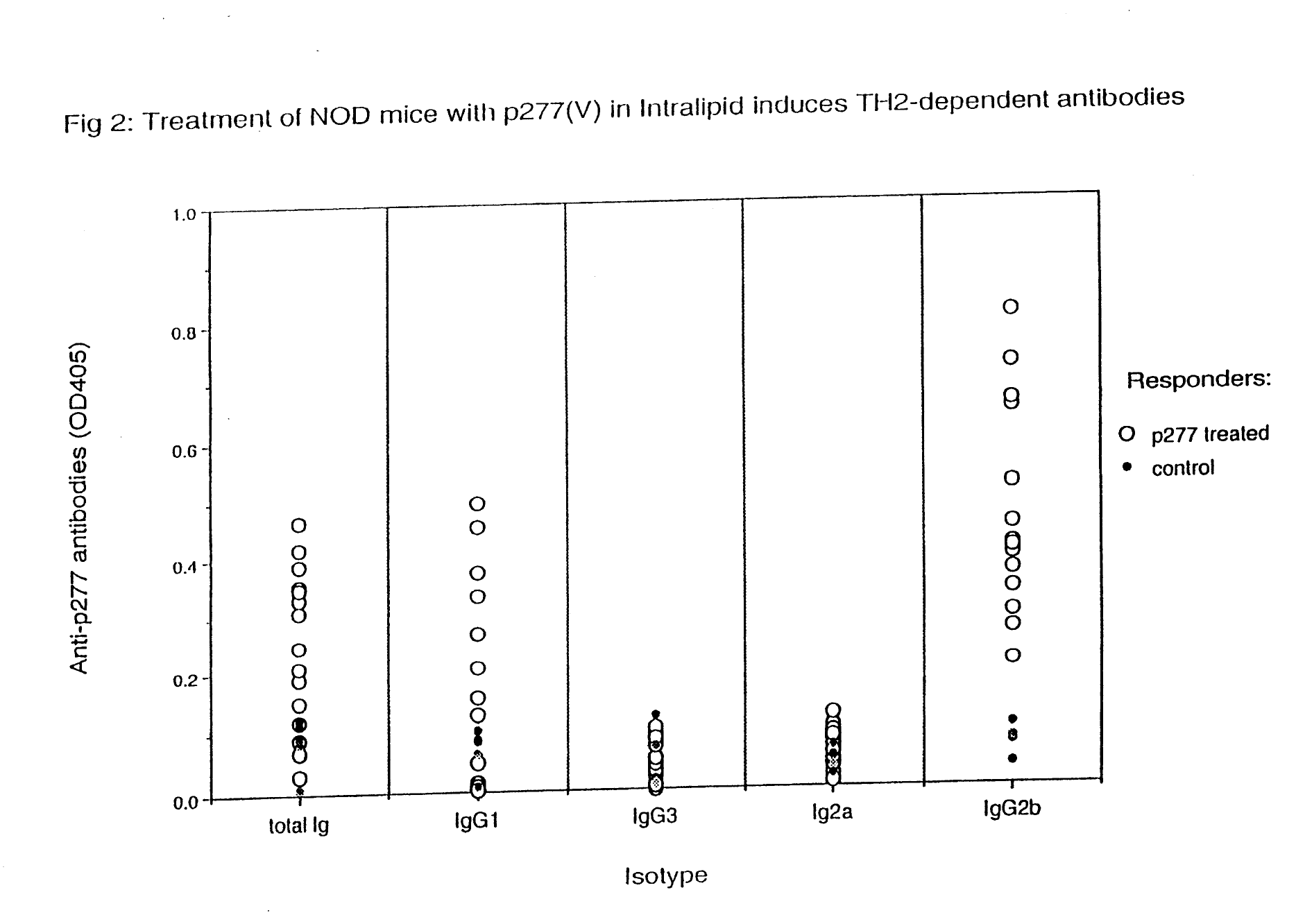

Groups of NOD mice, 3 months old, were treated with p277(Val⁶-Val¹¹) or with PBS in oil as described in Example 2. The sera of individual mice were assayed for the isotypes of their antibodies to p277(Val⁶-Val¹¹) after treatment (12-15 mice per group). The antibody isotypes were detected using an ELISA assay with isotype-specific developing antibody reagents (Southern Biotechnology Associates, Birmingham, Ala. ). The results are shown in FIG. 2, wherein: Antibodies to p277(Val⁶-Val¹¹) in control-treated NOD mice-open circles; in p277(Val⁶-Val¹¹)-treated mice-closed circles. The columns in each experiment show results from equal numbers of mice; an apparent reduction in numbers of circles is caused by superimposition.

Analysis of the antibody isotypes of the anti-p277 antibodies developing after treatment showed them to be exclusively of the IgG1 and IgG2b classes, dependent on TH2 T cells producing IL-4 (Snapper et al., 1993a) and possibly TGF/β (Snapper et al., 1993b). There were no TH1-type IgG2a antibodies induced by p277(Val⁶-Val¹¹) therapy. The development of antibodies to the specific peptide used in treatment is a sign that the autoimmune T-cell responses have shifted from a damaging inflammatory mode called TH1to a TH2 T-cell response that produces innocuous antibodies and suppresses inflammation and tissue damage (Rabinovitch, 1994).

### Example 4

Peptide p277(Val⁶-Val¹¹)/INTRALIPID® Therapy Induces a Specific Switch in the Cytokine Profile

To confirm the concept of a cytokine switch, the cytokines produced by the T cells reactive to the p277(Val⁶-Val¹¹) in the p277(Val⁶-Val¹¹)/Intralipid-treated and control mice were assayed. Concanavalin A (ConA), a T-cell mitogen, was used to activate total splenic T-cells as a control.

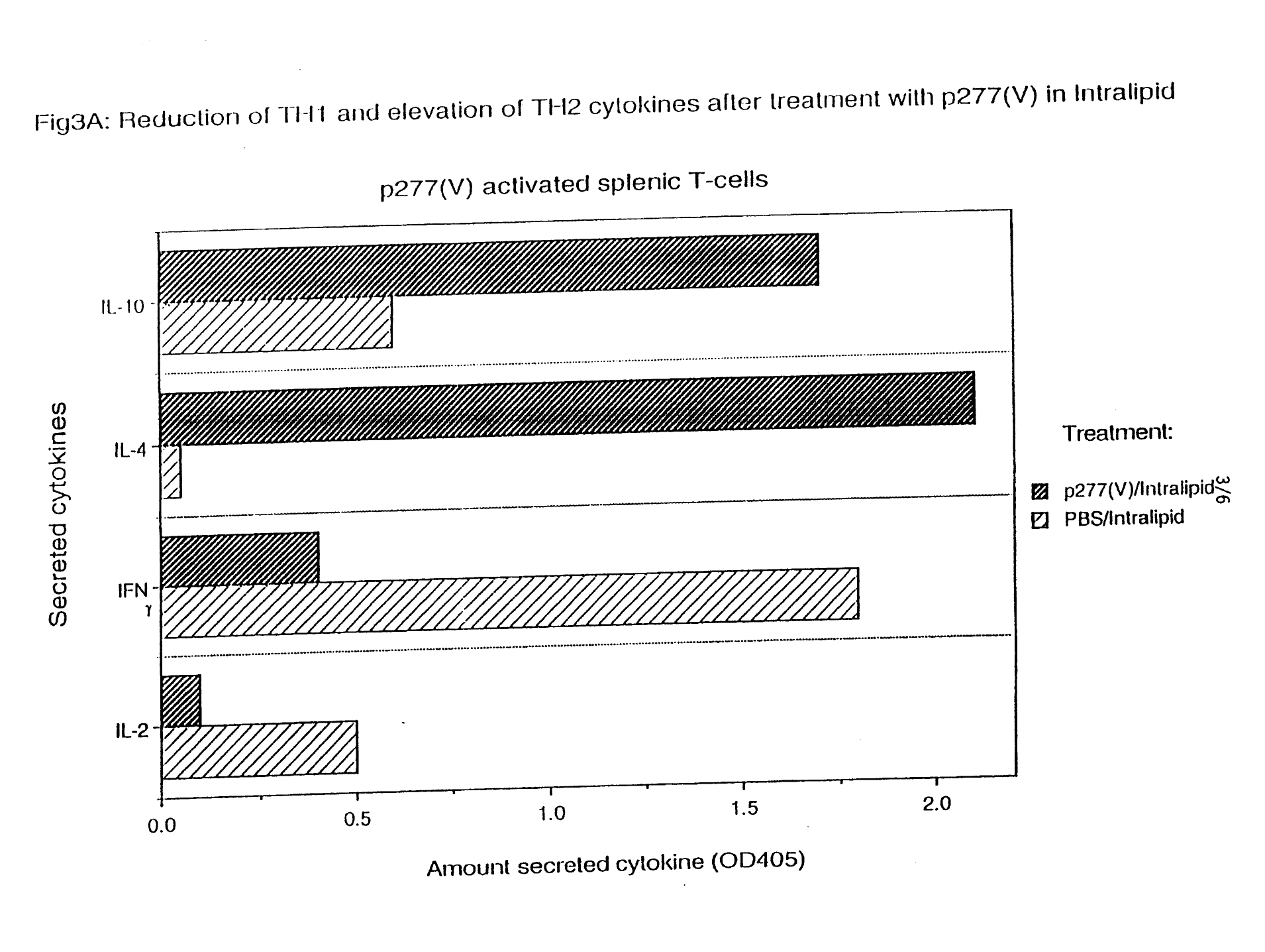

Groups of 10 NOD mice, 3 months old, were treated with p277(Val⁶-Val¹¹) in Intralipid (closed bars) or with PBS in Intralipid (open bars; see Example 2). Five weeks later, the spleens of the mice were removed and the spleen cells were pooled. The spleen cells were incubated-with Con A or p277(Val⁶-Val¹¹) for 24 h (for IL-2 and IL-4 secretion) or for 48 h (for IL-10 and IFN7 secretion). The presence of the cytokines in the culture supernatants was quantitated by ELISA, using Pharmingen paired antibodies according to the Pharmingen cytokine ELISA protocol. Pharmingen recombinant mouse cytokines were used as standards for calibration curves. Briefly, flat-bottom 96-well microtiter plates were coated with rat anti-mouse cytokine mAbs for 18 h at 4° C., and the culture supernatants or recombinant mouse cytokines were added for 18 h at 4° C. The plates were washed, and biotinylated rat anti mouse cytokine mAbs were added for 45 min at room temperature, then extensively washed, and avidin-alkaline phosphatase was added. The plates were washed, a chromogen substrate (p nitrophenylphosphate) was added and samples were read at 405 nm in an ELISA reader. The results are shown in FIG. 3. The concentrations of cytokines are shown as the OD readings. *P<0.01.

FIG. 3A shows that the spleen cells of control mice secreted both IL-2 and IFNγ upon incubation with p277(Val⁶-Val¹¹). In contrast, the p277(Val⁶-Val¹¹)-treated mice produced significantly less (P<0.01) IL-2 and IFNγ in response to incubation with peptide p277(Val⁶-Val¹¹). This reduction in TH1 cytokines was specific; the p277 (Val⁶-Val¹¹)-treated mice maintained their IL-2 and IFNγ cytokine responses to ConA (FIG. 3B). FIGS. 3A and 3B show the amounts of IL-10 and IL-4 produced by the spleen cells of the mice. The control mice produced very little IL-4 or IL-10 in response to p277(Val⁶-Val¹¹) or Con A. In contrast, there was a significant increase in IL-10 and IL-4 in response only to p277 (Val⁶-Val¹¹) and only in the p277 (Val⁶-Val¹¹)/Intralipid treated mice (P<0.01). A decrease in IL-2 and IFNγ coupled with an increase in IL-10 and IL-4 confirms the shift from TH1 like behavior to TH2-like behavior. Such a shift might help explain both a decline in T-cell proliferation to p277 shown previously by the inventors (Elias et al., 1991) and the appearance of IgG1 and IgG2b antibodies to p277(Val⁶-Val¹¹) according to the present invention.

### Example 5

Spontaneous T-Cell Proliferative Responses to p277(Val⁶-Val¹¹) is Reduced By p277 (Val⁶-Val¹¹) Therapy

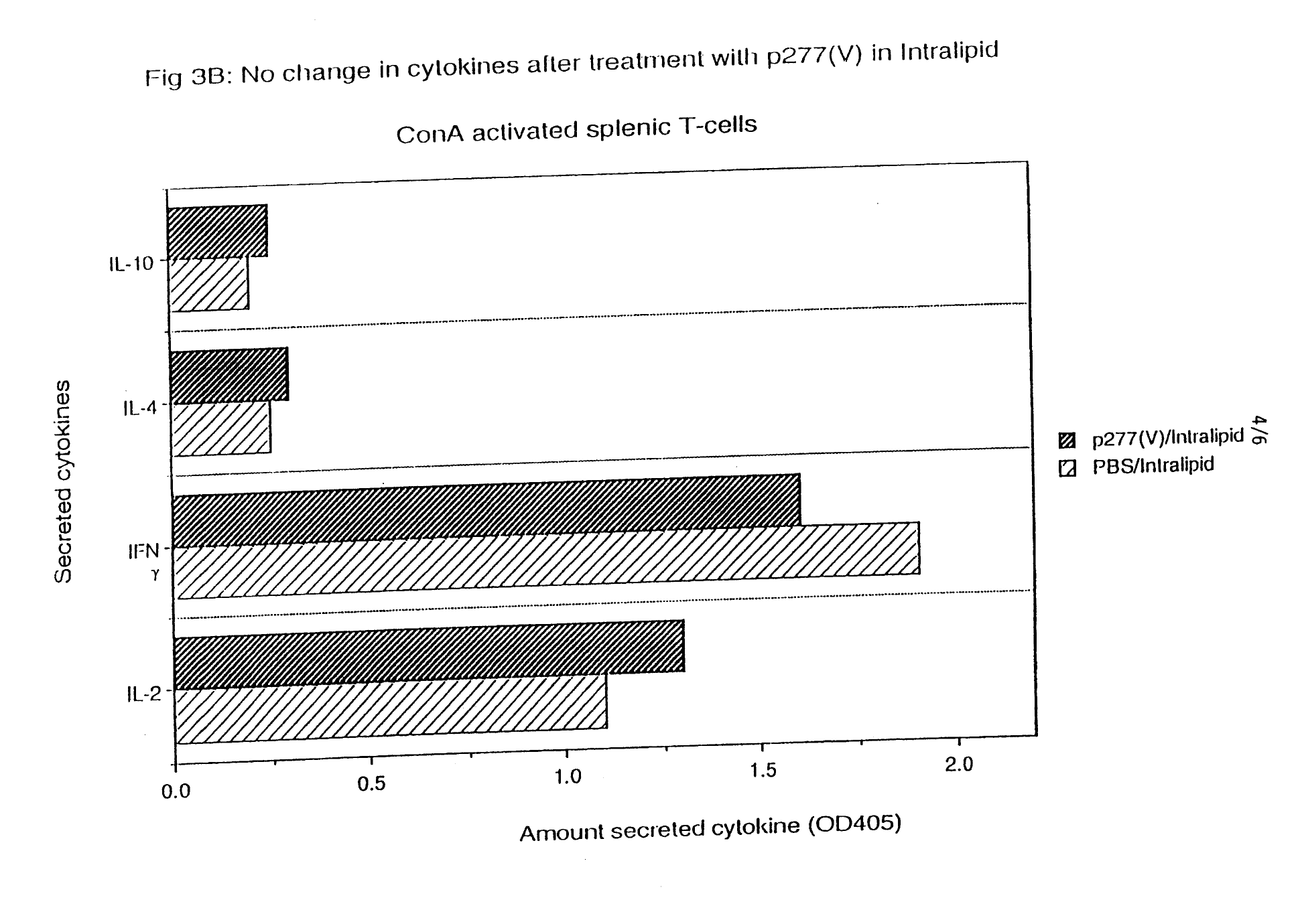

Groups of female mice of the NOD/Lt strain were treated at the age of 3 months with 100 μg of peptide p277(Val⁶-Val¹¹) in INTRALIPID or with PBS mixed with Intralipid, sc in the back. Five weeks later, the spleens of the mice were removed and the T-cell proliferative responses were assayed in vitro to the T-cell mitogen Con A (1.25 μg/ml) or to p277(Val⁶-Val¹¹) (10 μg/ml) using a standard assay. The results are shown in FIG. 4, wherein: Con A-black striped bars; p277(Val⁶-Val¹¹)-gray bars. The T-cell responses were detected by the incorporation of [³H] thymidine added to the wells in quadruplicate cultures for the last 18 hours of a 3-day culture. The stimulation index (SI) was computed as the ratio of the mean cpm of test cultures to the mean cpm of antigen-containing wells to control wells cultured without antigens or Con A. The standard deviations from the mean cpm were always less than 10%.

As shown in FIG. 4, the control mice tested with PBS/Intralipid showed T-cell proliferative responses to both p277(Val⁶-Val¹¹) and to the T-cell mitogen Con A. In contrast, the mice treated with p277(Val⁶-Val¹¹) in Intralipid showed a decrease in T-cell proliferative reactivity to p277(Val⁶-Val¹¹) but no decrease to Con A. Thus the beneficial effect of p277(Val⁶-Val¹¹) peptide therapy is caused not by inactivating the autoimmune response, but by activating the autoimmunity into a different cytokine mode of behavior (Cohen, 1995). Regulation of destructive autoimmunity is programmed within the immune system (Cohen, 1992); it need only be activated by a suitable signal which requires the peptide together with the lipid vehicle; neither the peptide alone or the lipid without the peptide are effective, as shown in Table 1. These results indicate that metabolizable lipid emulsions may be use defectively as vehicles for therapy of autoimmune diseases. Each disease will require its own specific peptide, but the metabolizable lipid emulsion can be used for the various therapies.

### Example 6

Administration of Peptide in Intralipid Affects Development of Experimental Autoimmune Encephalomyelitis

Experimental autoimmune encephalomyelitis (EAE) is an experimental autoimmune disease of animals that is thought to model aspects of multiple sclerosis (Zamvil and Steinman, 1990). EAE can be induced in susceptible strains of rats, such as the Lewis rat, by immunization to myelin basic protein (MBP) in complete Freund's adjuvant (CFA), an emulsion of mineral oil containing killed Mycobacteria. The disease develops about 12 days after immunization and is characterized by paralysis of various degrees due to inflammation of the central nervous system. The paralysis can last up to 6 or 7 days and the rats usually recover unless they die during the peak of their acute paralysis. EAE is caused by T cells that recognize defined determinants of the MBP molecule. The major MBP determinant in the Lewis rat is composed of the peptide sequence 71-90 (Zamvil and Steinman, 1990).

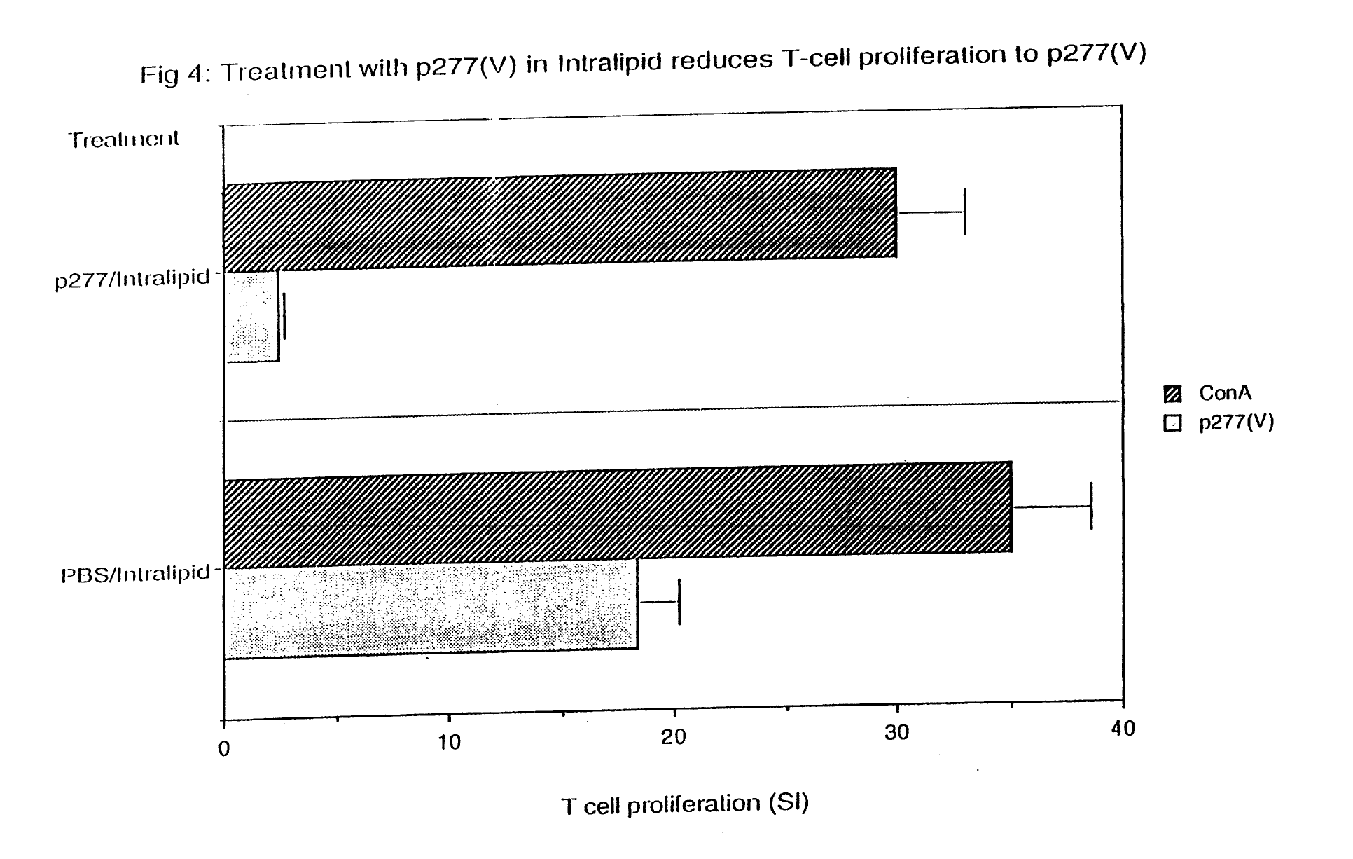

We therefore performed an experiment to test whether administration of the encephalitogenic MBP peptide p71-90 in IFA could also inhibit the development of EAE. FIG. 5 shows that the administration of p71-90 in IFA 14 days before the induction of EAE led to a significant decrease in the maximal degree of paralysis compared to the control treatment with PBS emulsified in IFA, which had no effect on the severity of the disease. Thus, p71-90 given in IFA affects EAE.

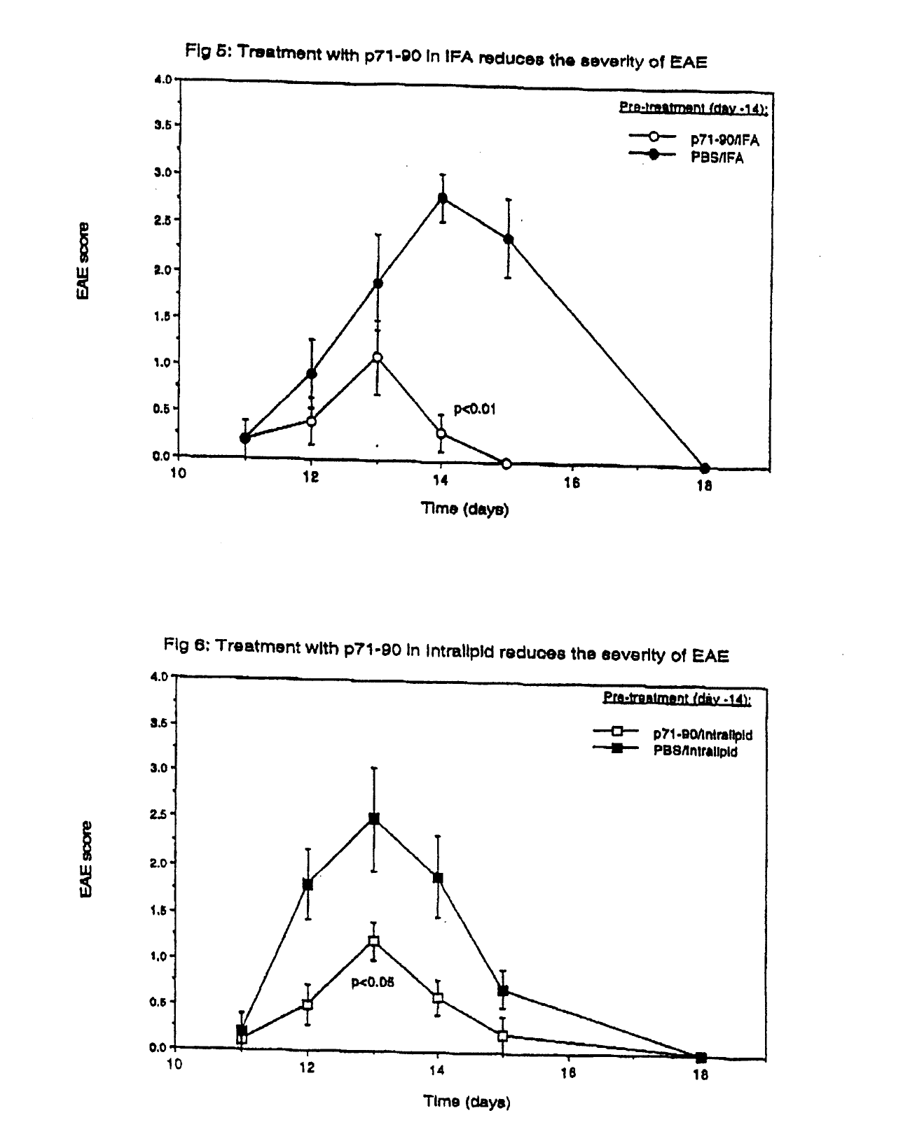

However, IFA cannot be administered, as stated above, to humans because it is not metabolizable in the body and causes local inflammation. We therefore treated Lewis rats with p71-90 in Intralipid. FIG. 6 shows the results. The rats that had received p71-90 in Intralipid developed significantly less paralysis than did the control rats treated with PBS/Intralipid. Therefore, it can be concluded that a relevant peptide such as p71-90 administered in Intralipid is capable of modulating EAE in rats. Hence, the effects of peptide/Intralipid treatment are not limited to only one peptide, in one species, or to only one autoimmune disease.

### Example 7

Effectiveness of New vs. Aged 10% Intralipid Emulsion

10% Intralipid emulsion was used to treat 12 week old NOD female mice with p277(Val⁶-Val¹¹) The emulsion was used either on the day the sealed bottle was opened, or 4 months later, after exposure to atmospheric air. The pH of the emulsion was tested at the time of preparing the peptide+emulsion for treatment. Aging was marked by a fall in pH from 8.2 to 6.7. In each experiment 10 mice were treated with the peptide+emulsion preparation, 10 mice received the emulsion alone, and 10 mice were untreated. The results are shown in Table 3.

TABLE 3

| | | Emulsion | Diabetes | Mortality |
|---|---|---|---|---|
| Group | Treatment | pH | (%) | (%) |
| 1 | peptide + emulsion | 8.2 | 20* | 10* |
| 2 | emulsion | ″ | 90 | 70 |
| 3 | peptide + emulsion | 6.7 | 60 | 40 |
| 4 | emulsion | ″ | 80 | 60 |
| 5 | untreated | — | 90 | 80 |

*p < 0.01

It can be seen that the placebo-treated mice (emulsion only, groups 2 and 4) and the untreated mice (group 5) developed a similar incidence of diabetes, 80-90% at 6 months of age. In contrast, treating the mice with peptide in the newly opened emulsion protected 80% of the mice from diabetes. However, using the “aged” emulsion only protected 40%. Therefore, the emulsion was chemically unstable after exposure to air, as shown by the marked decrease in pH value. This change is relevant to its biological activity. Hence, the Intralipid is a biologically active carrier whose functional properties depend on the pH and not only on the presence of inert lipid.

### Example 8

Reduction or Inhibition of β-cell Destruction in Patients Diagnosed with Type 1 Diabetes

We screened men, aged 16-55 years, who were consecutively diagnosed as having type 1 diabetes at the Endocrine Clinic of the Hadassah University Hospital. Although a phase I study of DiaPep277 injection in 16 adults with long-term diabetes showed no toxic effects at doses up to 2-5 mg per injection (data not shown), we excluded young children and women to obtain additional safety data in adults without a risk of pregnancy. Inclusion criteria were: presentation with acute hyperglycemia and ketonuria; a body-mass index of 28 kg/m² or less; no family history of type 2 diabetes; the presence of autoantibodies to glutamic acid decarboxylase; diabetes of less than 6 months' duration; residual β-cell function detected by a basal C peptide concentration of more than 0.1 nmol/L; and compliance with diet and insulin treatment with well controlled diabetes for at least 2 weeks. The patients were free of other diseases, and their informed consent was obtained.

We randomly assigned patients, with masking, to treatment with a preparation of p277 in oil (DiaPep277, Peptor Ltd., Rehovot, Israel) or placebo. Randomization was done by the contract research organization, in blocks of four. The randomization list was generated by the computer program RANCODE (version 3.6).

Peptide p277 was synthesized under the regulations of Good Manufacturing Practice by Peptor Ltd., 1 mg peptide and 40 mg mannitol (as a filler) in 0.5 ml was administered as DiaPep277 subcutaneously in a vehicle composed of a 10% preparation of a vegetable oil approved for human injection (LIPOFUNDINC® 10%, B Braun, Melsungen, Germany). The sequence of peptide p277 in DiaPep277 is VLGGGVALLRVIPALDSLTPANED, residues 437-460 of the human hsp 60 molecule. To stabilize DiaPep 277 without affecting its immunological properties we substituted valine for the cysteine residues at positions 442 and 447. The placebo was mannitol (40 mg) in the vehicle. Every patient received three injections: at enrolment, 1 month, and 6 months.

We followed up the patients for 10 months to allow time for loss of most of the capacity to produce C-peptide in response to glucagon stimulation. The identity of the groups was unknown to the patients, their physicians, or the staff doing laboratory testing, but it was known to a safety committee, who followed up on the patients for adverse effects, and to professional statisticians, who tabulated the data. The study was approved by the institutional review board and by the National Committee for Human Trials of the Israel Ministry of Health.

Endpoint Assessments

To assay the functional β-cell mass, the primary endpoint, C-peptide concentration in the morning, 10-12 h after the last insulin dose, as the fasting basal concentration, and 2 min, 6 min, 10 min, and 20 min after stimulation of the patient with intravenous glucagon (1 mg) was measured by a standard assay. The highest concentration was used as the value for analysis.

The patients' doctors prescribed the amounts of insulin required to control each patient's blood glucose concentration according to accepted standards, and the amount of insulin per kg bodyweight was calculated from the patient's treatment diary. In addition to standard blood tests to detect possible toxic effects, blood samples were tested for hemoglobin Alc (glycosylated hemoglobin) as a measure of the general control of hyperglycemia.

The cytokine phenotype of the T-cell reactivity to hsp60 and to peptide p277 was measured in vitro with a quantitative ELISpot assay. In this assay, peripheral blood T cells are stimulated by incubation in vitro with the antigen (10 μg/mL), and the numbers of T cells producing various cytokines are enumerated by counting spots in a cytokine capture assay. Interferon γ, a Th1 cytokine, and interleukins 4, 10, and 13, Th2 cytokines, were measured. T-cell responses were also measured to bacterial recall antigens Mycobacterium tuberculosis (purified protein derivative; PPD) and tetanus toxoid as described.

Of 47 patients screened, 35 were eligible, 18 were assigned DiaPep277 and 17 placebo. By the end of the follow-up period, four patients had been lost to follow-up (one was excluded for drug use and three refused to undergo the glucagon stimulation assay). The DiaPep277 and placebo groups were similar in terms of age (29.3 [SD 11.9] vs 23.1 [6.9] years), body mass index (22.1 [2.9] vs 21.9 [2.7] kg/m²), duration of disease (14.5 [9.9] vs 12.6 [6.6] weeks), baseline C-peptide concentration (0.44 [0.30] vs 0.53 [0.40] nmol/L), and insulin requirements on entry (0.35 [0.14] vs 0.37 [0.19] U/kg).

Patients on placebo showed a progressive loss of glucagon-stimulated C-peptide, indicating a progressive, cumulative loss of β cells with time. Indeed, the rapid loss of C-peptide supports the diagnosis of type I diabetes. The group of patients assigned DiaPep277, by contrast, maintained their production of C-peptide after glucagon stimulation. The differences between the DiaPep277 and placebo groups were significant at 7 months and 10 months of follow-up (0.92 [SD 0.25] vs 0.35 [0.22] n-mol/L, p=0.043, at 7 months; 0.93 [0.35] vs 0.26 [0.11] nmol/L, p=0.039 at 10 months). There was a positive between C-peptide concentrations at entry and at 2 months in both groups (correlation coefficient 0.65 in the DiaPep 277 group, 0.70 in the placebo group). At 10 months, however, the correlation coefficient of the DiaPep277 group was 0.82 and that of the placebo group only 0.02. Thus, the individuals with higher C-peptide concentrations at the time of initiation of DiaPep277 treatment showed better preservation of C-peptide concentration 10 months later.

The concentrations of hemoglobin A1c seen over 10 months in both groups were around 7% throughout the study. This value indicates that patients in both groups received adequate treatment. Thus, any differences between the groups could be attributed to the effects of treatment, not to any difference in metabolic control.

The DiaPep277 group required less exogenous insulin to maintain adequate control than did the placebo group. The difference at 10 months was significant (p=0.042).

T-cell responses to human hsp60, peptide p277, and bacterial antigens were assessed at 10 months. Patients on placebo showed more interferon γ (p=0.041) and less interleukin 13 (p=0.048) in response to hsp60 than to p277. Thus, hsp 60 seems to activate more of a Th1 response than does p277 in controls. Compared with the placebo group, patients assigned DiaPep277 produced less interferon γ (p=0.04) and more interleukin 10 (p=0.03) and interleukin 13 (p=0.04) in response to hsp60; the increase in interleukin 4 was not significant (p=0.14). In response to p277, the DiaPep277 patients produced more interleukin 10 (p=0.01) and interleukin 13 (p=0.02) than patients on placebo; the differences in interleukin 4 and interferon γ were not significant (p=0.13 and p=0.12, respectively).

The induction of antibodies to p277 by DiaPep277 treatment could not be measured because many of the patients tested positive for such antibodies before they received treatment (data not shown). There were no great differences in the T-cell proliferation and cytokine responses between the groups to PPD or tetanus toxoid (data not shown). There was a positive correlation between the amount of interleukin 13 produced in response to hsp60 and the mean concentration of C-peptide at 10 months in the DiaPep277-group patients (correlation coefficient 0.75).

No adverse effects of treatment were noted, except for slight redness at the injection site in four patients, which resolved within 24-48 h without treatment.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present application. Therefore, such adaptations and modifications are intended to be within the meaning and range of equivalents of the disclosed embodiments, based on the teaching and guidance presented herein. It is to be understood that the phraseology of the present specification is to be interpreted by the skilled artisan in light of the teachings and guidance presented herein, in combination with the knowledge of one of ordinary skill in the art.

### Example 9

Preferred Formulations (Containing p277), Effective at Reducing the Incidence or Severity of IDDM

The following preferred specific lipid formulations have been effectively used as vehicles for peptides to effectively reduce the incidence or severity of IDDM:

Formula 1: 10% soybean oil; 0.8% phospholipids (egg); 2.5% glycerol; 0.05% alpha-Tocopherol; and sterile water. Sodium oleate is also added to adjust pH;

Formula 2: 20% soybean oil; 1.2% phospholipids (egg); 2.5% glycerol; 0.05% alpha-Tocopherol; and sterile water. Sodium oleate is also added to adjust pH;

Formula 3: 10% soybean oil; 0.6%-1.2% phospholipids (egg yolk); 2.2% glycerol; and sterile water. Sodium hydroxide was added to adjust the pH of the formulation; and

Formula 4: 20% soybean oil; 1.2% phospholipids (egg yolk); 2.2% glycerol; and sterile water. Sodium hydroxide was added to adjust the pH of the formulation.

### References

1. Allegretta, M., Nicklas, J. A., Sriram, S. and Albertini, R. J., Science 247:718-721 (1990).

2. Banchereau, J. and Rybak, M. E., in: The Cytokine Handbook, 2nd Ed., A, Thompson Ed. Academic Press New York, pp 99 (1994).

3. Cohen, I. R., Immunology Today 13:490-494 (1992).

4. Cohen, I. R., in: Selective Immunosuppression: Basic Concepts and Clinical Applications (Eds. Adorini, L., Capra, D. J., Waldmann, H.) Chem. Immunol Karger, Basel, 60:150-60 (1995).

5. Kristiansen, T., Sparrman, M. and Heller, L., J. Biosci 5 (suppl. 1):149-155 (1983).

6. Liblau, R. S., Singer, S. M and McDevitt, H. O., Immunology Today 16:34 38 (1995).

7. Moore, K. V., O'Garra, A., de Waal Malefyt, R., Vieira, and Mosmann, T. R., Ann. Rev. Immunol. 711:165-190 (1993).

8. Mosmann, T. R. and Coffman R. L., Ann. Rev Immunol. 7:145-173 (1989).

9. Ota, K., Matsui, M., Milford, E. L., Mackin, G. A., Weiner, H. I. and Hafler, D. A., Nature 346:183-187 (1990).

10. Rabinovitch, A., Sorensen, O., Sua-Pinzon, W. K., Rajotta, R. V. and Bleakley, R. C., Diabetologia 37:833-837 (1994).

11. Romagnani, S., Ann. Rev. Immunol. 12:227-257 (1994).

12. Snapper, C. M. and Mond, J. J., Immunology Today 14:15-17 (1993).

13. Snapper, C. M., Waegell, W., Beernink, H. and Dasch, J. R., J. Immunol. 151:4625 36 (1993).

14. Zarnvil, S. S. and Steinman, L., Ann. Rev. Immunol 8:579-621 (1990).

1. A composition of an antigen and a carrier, wherein the antigen is recognized by inflammatory T cells associated with the pathogenesis of diabetes and the carrier is a metabolizable lipid emulsion consisting essentially of 5-25% triglycerides of plant and/or animal origin, about 0.1-3% phospholipids of plant and/or animal origin, about 1.5-4.5% osmo-regulator and sterile water to complete to 100%, said composition induces a TH1→TH2 shift in the cytokines produced by said T cells.

2. The composition of claim 1, wherein the peptide is peptide p277 (residues 437-460 of SEQ ID NO: 1) or an analog thereof recognized by the inflammatory T cells associated with the pathogenesis of diabetes.

3. The composition of claim 2, wherein the analog is peptide p277 (Val⁶-Val¹¹) (SEQ ID NO: 4).

4. The composition according to claim 1, wherein the triglycerides and phospholipids are present in a weight ratio of between about 3:1 and 50:1 and the emulsion further comprises up to 0.05% antioxidant.

5. The composition according to claim 1, wherein the emulsion contains about 10-20% triglycerides, about 0.6-1.2% phospholipids, about 2.2-2.5% osmo-regulator and sterile water.

6. The composition according to claim 5, wherein the emulsion contains about 10% soybean oil, about 0.6%-1.2% egg-yolk phospholipids, about 2.2-2.5% glycerol and sterile water.

7. The composition according to claim 5, wherein the emulsion contains about 10% soybean oil, about 0.8% egg-yolk phospholipids, about 2.5% glycerol and sterile water.

8. The composition according to claim 1, wherein the triglycerides are derived from plant origins.

9. The composition according to claim 8, wherein the triglycerides are derived from soybean, cottonseed, coconut, or olive plants.

10. The composition according to claim 1, wherein the phospholipids are derived from animal origin.

11. The composition according to claim 10, wherein the phospholipids are derived from egg yolk or bovine serum.

12. The composition according to claim 1, wherein the osmo-regulator is glycerol, sorbitol or xylitol.

13. A method of reducing the incidence or severity of insulin dependent diabetes mellitus (IDDM) which comprises administering to a subject suffering from IDDM, a pharmaceutical composition of an antigen and a carrier, wherein the antigen is recognized by inflammatory T cells associated with the pathogenesis of diabetes and the carrier is a lipid emulsion, said composition induces a TH1→TH2 shift in the cytokines produced by said T cells and is administered in an amount which is effective to reduce the severity of symptoms associated with IDDM.

14. The method of claim 13, wherein the antigen is peptide p277 (residues 437-460 of SEQ ID NO: 1) or an analog thereof recognized by the inflammatory T cells associated with the pathogenesis of diabetes.

15. The method of claim 14, wherein the analog is peptide p277 (Val⁶-Val¹¹) (SEQ ID NO: 4).

16. The method of claim 13, wherein the subject is human.

17. The method according to claim 13, wherein the composition is administered in an amount sufficient to cause a decrease in IL-2 or IFN-γ TH1 cell cytokine response and an increase in IL-4 or IL-10 TH2 cell cytokine response.

18. The method according to claim 13, wherein the emulsion is a lipid emulsion comprising about 5-25% triglycerides of plant and/or animal origin, about 0.1-3% phospholipids of plant and/or animal origin, about 1.5-4.5% osmo-regulator and sterile water.

19. The method according to claim 18, wherein the triglycerides and phospholipids are present in a weight ratio of between about 3:1 and 50:1 and the emulsion further comprises up to 0.05% antioxidant.

20. The method according to claim 18, wherein the emulsion contains about 10-20% triglycerides, about 0.6-1.2% phospholipids, about 2.2-2.5% osmo-regulator and sterile water.

21. The method according to claim 20, wherein the emulsion contains about 10% soybean oil, about 0.6-1.2% egg-yolk phospholipids, about 2.2-2.5% glycerol and sterile water.

22. The method according to claim 21, wherein the emulsion contains about 10% soybean oil, about 0.8% egg-yolk phospholipids, about 2.5% glycerol and sterile water.

23. A method of reducing or inhibiting β-cell destruction which comprises administering to a subject suffering from β-cell destruction, a pharmaceutical composition of an antigen and a carrier, wherein the antigen is recognized by inflammatory T cells associated with the pathogenesis of β-cell destruction and wherein the carrier is a lipid emulsion, said composition induces a TH1→TH2 shift in the cytokines produced by said T cells and is administered in an amount which is therapeutically effective to reduce or inhibit β-cell destruction in said subject.

24. The method of claim 23, wherein the peptide is peptide p277 (residues 437-460 of SEQ ID NO: 1) or an analog thereof.

25. The method of claim 24, wherein the analog is peptide p277 (Val⁶-Val¹¹) (SEQ ID NO: 4).

26. The method of claim 23, wherein the subject is human.

27. The method according to claim 23, wherein the composition is administered in an amount sufficient to cause a decrease in IL-2 or IFN-γ T-cell cytokine response and an increase in IL-4 or IL-10 T-cell cytokine response.

28. The method according to claim 23, wherein the emulsion is a fat emulsion that comprises about 5-25% triglycerides of plant and/or animal origin, about 0.1-3% phospholipids of plant and/or animal origin, about 1.5-4.5% osmo-regulator and water.

29. The method according to claim 28, wherein the triglycerides and phospholipids are present in a weight ratio of between about 3:1 and 50:1 and the emulsion further comprises up to 0.05% antioxidant.

30. The method according to claim 28, wherein the emulsion contains about 10-20% triglycerides, about 0.6-1.2% phospholipids, about 2.2-2.5% osmo-regulator and water.

31. The method according to claim 30, wherein the emulsion contains about 10% soybean oil, about 0.6-1.2% egg-yolk phospholipids, about 2.2-2.5% glycerol and sterile water.

32. The method according to claim 30, wherein the emulsion contains about 10% soybean oil, about 0.8% egg-yolk phospholipids, about 2.5% glycerol and sterile water.

33. A method of reducing the incidence or severity of insulitis which comprises administering to a subject a pharmaceutical composition of an antigen and a carrier, wherein the antigen is recognized by inflammatory T cells associated with the pathogenesis of insulitis and wherein the carrier is a lipid emulsion, said composition induces a TH1→TH2 shift in the cytokines produced by said T cells and is administered in an amount which is effective to reduce the severity of insulitis.

34. The method of claim 23, wherein the peptide is peptide p277 (residues 437-460 of SEQ ID NO: 1) or an analog thereof.

35. The method of claim 24, wherein the analog is peptide p277 (Val⁶-Val¹¹) (SEQ ID NO: 4).

36. The method of claim 33, wherein the subject is human.

37. The method according to claim 33, wherein the composition is administered in an amount sufficient to cause a decrease in IL-2 or IFN-γ T-cell cytokine response and an increase in IL-4 or IL-10 T-cell cytokine response.

38. The method according to claim 33, wherein the emulsion is a fat emulsion that comprises about 5-25% triglycerides of plant and/or animal origin, about 0.1-3% phospholipids of plant and/or animal origin, about 1.5-4.5% osmo-regulator and water.

39. The method according to claim 38, wherein the triglycerides and phospholipids are present in a weight ratio of between about 3:1 and 50:1 and the emulsion further comprises up to 0.05% antioxidant.

40. The method according to claim 38, wherein the emulsion contains about 10-20% triglycerides, about 0.6-1.2% phospholipids, about 2.2-2.5% osmo-regulator and water.

41. The method according to claim 40, wherein the emulsion contains about 10% soybean oil, about 0.6-1.2% egg-yolk phospholipids, about 2.2-2.5% glycerol and sterile water.

42. The method according to claim 40, wherein the emulsion contains about 10% soybean oil, about 0.8% egg-yolk phospholipids, about 2.5% glycerol and sterile water.

43. A method of reducing the incidence or severity of latent autoimmune diabetes in adults (LADA) which comprises administering to a subject a pharmaceutical composition of an antigen and a carrier, wherein the antigen is recognized by inflammatory T cells associated with the pathogenesis of LADA and wherein the carrier is a lipid emulsion, said composition induces a TH1→TH2 shift in the cytokines produced by said T cells and is administered in an amount which is effective to reduce the severity of LADA.

44. The method of claim 43, wherein the peptide is peptide p277 (residues 437-460 of SEQ ID NO: 1) or an analog thereof.

45. The method of claim 44, wherein the analog is peptide p277 (Val⁶-Val¹¹) (SEQ ID NO: 4).

46. The method of claim 43, wherein the subject is human.

47. The method according to claim 43, wherein the composition is administered in an amount sufficient to cause a decrease in IL-2 or IFN-γ T-cell cytokine response and an increase in IL-4 or IL-10 T-cell cytokine response.

48. The method according to claim 43, wherein the emulsion is a fat emulsion that comprises about 5-25% triglycerides of plant and/or animal origin, about 0.1-3% phospholipids of plant and/or animal origin, about 1.5-4.5% osmo-regulator and water.

49. The method according to claim 48, wherein the triglycerides and phospholipids are present in a weight ratio of between about 3:1 and 50:1 and the emulsion further comprises up to 0.05% antioxidant.

50. The method according to claim 48, wherein the emulsion contains about 10-20% triglycerides, about 0.6-1.2% phospholipids, about 2.2-2.5% osmo-regulator and water.

51. The method according to claim 50, wherein the emulsion contains about 10% soybean oil, about 0.6-1.2% egg-yolk phospholipids, about 2.2-2.5% glycerol and sterile water.

52. The method according to claim 50, wherein the emulsion contains about 10% soybean oil, about 0.8% egg-yolk phospholipids, about 2.5% glycerol and sterile water.


SEQUENCE LISTING (4 sequences)

SEQ ID NO: 1 - protein, 573 aa
MLRLPTVFRQMRPVSRVLAPHLTRAYAKDVKFGADARALMLQGVDLLADAVAVTMGPKGRTVIIEQGWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 2 - protein, 24 aa
VLGGGVALLRCIPALDSLTPANED

SEQ ID NO: 3 - protein, 24 aa
VLGGGCALLRVIPALDSLTPANED

SEQ ID NO: 4 - protein, 24 aa
VLGGGVALLRVIPALDSLTPANED